(19) United States
(12) Patent Application Publication
(10) Pub. No.: US 2011/0281813 A1
(43) Pub. Date: Nov. 17, 2011

(54) METHOD OF TREATING C-KIT POSITIVE RELAPSED ACUTE MYELOID LEUKEMIA

(75) Inventors: Anjali Advani, Cleveland, OH (US); Matt Kalaycio, Cleveland, OH (US)

(73) Assignee: CLEVELAND CLINIC, Cleveland, OH (US)

(21) Appl. No.: 13/132,015

(22) PCT Filed: Dec. 3, 2009
(86) PCT No.: PCT/US2009/066477

§ 371 Date: Aug. 4, 2011

Related U.S. Application Data
(60) Provisional application No. 61/119,778, filed on Dec. 4, 2008.

(51) Int. Cl. A61K 31/704; A61P 35/02

(52) U.S. Cl. 514/34

(57) ABSTRACT

The present invention relates to a method of treating c-kit positive relapsed acute myeloid leukemia (AML) in a human patient population using a combination comprising a c-kit inhibitor, daunorubicin and cytarabine.

The present invention relates to a method of treating c-kit positive relapsed acute myeloid leukemia (AML) in a human patient population using a combination comprising a c-kit inhibitor, daunorubicin and cytarabine.

C-kit is expressed on more than 10% of blasts in 95% of relapsed AMLs. Imatinib mesylate is a c-kit inhibitor and has single agent activity in relapsed/ refractory AML. For instance, the treatment of AML with Imatinib was described by J Cortes, F Giles, S O'Brien et al, Results of Imatinib mesylate in patients with refractory or recurrent acute myeloid leukemia, high-risk myelo-dysplastic syndrome and myeloproliferative disorders, Cancer 97, 2003, 2760-2766; T Kindler, F Breitenbuecher, A Marx et al, Efficacy and safety of Imatinib in adult patients with c-kit positive acute myeloid leukemia; Blood 2004; 103; 3644-54.

It was now found that patients with c-kit+ relapsed AML can be successfully treated with a combination comprising Imatinib mesylate, daunorubicin and cytarabine. Hence, the present invention provides a method for treating c-kit positive relapsed acute myeloid leukemia AML in a human patient in need thereof, comprising co-administration to said patient, e.g., concomitantly or in sequence, of a therapeutically effective amount of a c-kit inhibitor, daunorubicin and cytarabine or, respectively, a pharmaceutically acceptable salt thereof.

In a further aspect, the present invention relates to the use of a combination comprising a c-kit inhibitor, daunorubicin and cytarabine for the manufacture of a medicament for the treatment of c-kit positive relapsed acute myeloid leukemia AML.

The expression “c-kit inhibitor” as used herein includes, but is not limited to, 4-(4-methylpiperazin-1-ylmethyl)-N-[4-methyl-3-(4-(pyridin-3-yl)pyrimidin-2-ylamino)phenyl]-benzamide (Imatinib) and 4-methyl-3-[[4-(3-pyridinyl)-2-pyrimidinyl]amino]-N-[5-(4-methyl-1H-imidazol-1-yl)-3-(trifluoromethyl)phenyl] benzamide (Nilotinib) and, respectively, the pharmaceutically acceptable salts thereof.

In a preferred the c-kit inhibitor employed is Imatinib. Imatinib is specifically disclosed in the patent applications U.S. Pat. No. 5,521,184, the subject-matter of which is hereby incorporated into the present application by reference. Imatinib can also be prepared in accordance with the processes disclosed in WO03/066613. For the purpose of the present invention, Imatinib is preferably applied in the form of its mono-mesylate salt. Imatinib mono-mesylate can be prepared in accordance with the processes disclosed in U.S. Pat. No. 6,894,051. Comprised by the present invention are likewise the corresponding polymorphs, e.g. crystal modifications, which are disclosed in U.S. Pat. No. 6,894,051.

In a further preferred embodiment of the method described herein the mono-mesylate salt of Imatinib is administered orally in dosage forms as described in U.S. Pat. No. 5,521,184, U.S. Pat. No. 6,894,051 or US 2005-0267125. The mesylate salt of Imatinib is marketed under the brand Glivec® (Gleevec®). A preferred oral daily dosage of Imatinib is 200-400 mg, in particular 300 mg/day, administered as a single dose or divided into multiple doses, such as twice daily dosing.

Using the combination as described herein employing Imatinib mesylate as c-kit inhibitor in one elderly patients not suitable for bone marrow transplantation (BMT) has remained in remission on for at least 574 days.

In a further preferred embodiment of the present invention, the c-kit inhibitor employed is Nilotinib. Nilotinib and the process for its manufacture are disclosed in WO 04/005281, which is hereby incorporated into the present application by reference. Pharmaceutically acceptable salts of Nilotinib are especially those disclosed in WO2007/015871. For the purpose of the present invention, Nilotinib is preferably applied in the form of its mono-hydrochloride mono-hydrate salt. WO2007/015870 discloses certain polymorphs of nilotinib and its pharmaceutically acceptable salts useful for the present invention.

In a further preferred embodiment of the method described herein the mono-hydrochloride salt of Nilotinib is administered orally in dosage forms as described in WO2008/037716. The mono-hydrochloride salt of Nilotinib is marketed under the brand Tasigna®. A preferred oral daily dosage of Nilotinib is 200-1200 mg, e.g. 800 mg, administered as a single dose or divided into multiple doses, such as twice daily dosing.

Daunorubicin and cytarabine (also known as ara C) are commonly used to treat AML. Both drugs can be administered, for instance by injection through a cannula inserted into the vein. Daunorubicine is. e.g. marketed in the form of its hydrochloride salt, e.g. under the brand Cerubidine™. In one embodiment of the invention, patients receive 45 mg/m² of Daunorubicin intravenous for 3 days and 100 mg/m²/day of cytarabine oer continuous infusion for 7 days (7+3).

In a preferred embodiment of the present invention, patients are treated not showing any nuclear phospho-STAT3 expression.

The structure of the active agents identified by generic or trade names may be taken from the actual edition of the standard compendium “The Merck Index” or from databases, e.g. Patents International (e.g. IMS World Publications). The corresponding content thereof is hereby incorporated by reference.

In particular, the present invention provides

(1) the use of a combination comprising a c-kit inhibitor, preferably selected from Imatinib and Nilotinib or a pharmaceutically acceptable salt thereof, respectively, daunorubicin and cytarabine for the manufacture of a medicament for the treatment of c-kit positive relapsed acute myeloid leukemia AML;
(2) the use of such combination characterized in that the c-kit positive relapsed acute myeloid leukemia AML is observed in patients, which do not show any nuclear P-STAT3 expression.
(3) a package comprising a combination comprising a c-kit inhibitor, daunorubicin and cytarabine together with instructions to use the combination for the treatment of c-kit positive relapsed acute myeloid leukemia AML, in particular in patients, which do not show any nuclear P-STAT3 expression.

Suitable clinical studies in human patients are, for example, open label non-randomized, studies in patients with c-kit positive relapsed acute myeloid leukemia. Such studies prove in particular superiority of the claimed method of treatment compared to treatments with one of the components of the treatment schedule alone. The beneficial effects on c-kit positive relapsed acute myeloid leukemia can be determined directly through the results of these studies or by changes in the study design which are known as such to a person skilled in the art.

### EXAMPLES

The following Example illustrates the invention described above, but is not, however, intended to limit the scope of the invention in any way. Other test models known as such to the person skilled in the pertinent art can also determine the beneficial effects of the claimed invention.

### Example 1

Phase 1 Trial of Imatinib Mesylate Combined with Daunorubicin (D) and Cytarabine (A) in Patients with C-kit+ Relapsed AML

Methods: All patients were treated at the Cleveland Clinic from 2003-2008. Cytogenetic (CG) risk was defined by CALGB criteria. Eligibility criteria included: relapsed AML (excluding acute promyelocytic leukemia (APL)), relapse more than 6 mo from induction tx, ECOG 0-2, ≧20% blasts c-kit+ (CD117+by flow cytometry), age≧18 yrs, not pregnant or lactating, creatinine ≦2 mg/dl, AST and ALT≦2×ULN (upper limits of normal), bilirubin≦2 mg/dl. The concurrent use of antimicrobials which significantly interact with Imatinib mesylate was prohibited. All patients received D 45 mg/m² intravenous for 3 days and A 100 mg/m²/d continuous infusion for 7 days (7+3). Imatinib mesylate was dose escalated using a standard 3×3 design. Six additional patients were then treated at the Maximum Tolerated Dose (MTD). Dose Levels were: −1 (300 mg), 1 (400 mg), 2 (600 mg), 3 (800 mg). Imatinib mesylate was administered with the first dose of chemotherapy and continued daily until disease progression, intolerance, off-study for another treatment (including alloBMT), or dose-limiting toxicity (DLT). A Day 14 bone marrow was performed. Patients with persistent leukemia, but ≧50% reduction in bone marrow blasts remained on protocol. These patients continued Imatinib mesylate and received 5 days of A (100 mg/m²/d) and 2 days of D (45 mg/m²/d). C-kit expression was determined by staining cells with anti-CD45-Per-CP and anti-CD117-PE. Using a CD45/orthogonal light scatter to isolate blasts, the mean fluorescent intensity (MFI) was calculated as the mean channel number (MCN) of the blasts/ MCN autofluorescence. Phosphorylated (P) STAT3 and STAT5 (nuclear and cytoplasmic) were assessed by immunohistochemistry on pre-treatment samples.

Results: Twenty-one patients have been enrolled: median age 47 yrs (range 24-75) and 48% were male. The average time from complete remission (CR) to relapse was 439 d (range 216-1100). CG risk: poor 14%, intermediate 71%, and good 14%. The median % of c- kit positive blasts was 89% (range 52-98). In patients expressing nuclear P-STAT3, 2-6% of the blasts stained positive. Eleven patients had no nuclear P-STAT3. No patients had cytoplasmic P-STAT3. At 400 mg (Dose Level 1), 2 out of 6 patients had a DLT, and both of these were ≧Grade 3 hepatotoxic. Therefore, subsequent patients were treated at Dose Level −1. In the expanded cohort at Dose Level −1, 1 patient had muscle cramps (Grade 3) and 1 patient had sudden vision loss. Both serious adverse events were thought to be Imatinib mesylate-related. One patient died during induction and was not evaluable for response. Eleven out of the 19 evaluable patients (58%) achieved a CR or complete remission without platelet recovery (CRp). Patients received Imatinib mesylate for a median of 23 days (range 9-574+days). Three discontinued Imatinib mesylate when they proceeded to alloBMT. Two patients continue on Imatinib mesylate and remain in a remission 574+and 52+days. There was no correlation between c-kit MFI or P-STAT5 expression and achievement of CR. Patients with no nuclear P-STAT3 had a higher CR rate (82%) than patients with P-STAT3 nuclear expression (33%) (p=0.18).

Conclusions: The MTD of Imatinib mesylate in combination with 7+3 was 300 mg. Since the hepatoxicity appeared to be reversible with discontinuation of Imatinib mesylate, it may be possible to escalate Imatinib mesylate doses further and re-challenge. PK/PD data to better understand the cause of this toxicity is pending. The CR rate is encouraging, and deserves further evaluation in a Phase 2 study. In particular, patients with no nuclear P-STAT3 had a CR rate of 82%; whereas, patients with nuclear P-STAT3 expression had a CR of 33%.

1. Method of treating c-kit positive relapsed acute myeloid leukemia (AML) in a human patient which does not show any nuclear P-STAT3 expression comprising administering to the human patient which does not show any nuclear P-STAT3 expression in need thereof a dose effective against AML of a combination comprising a c-kit inhibitor, daunorubicin and cytarabine or a pharmaceutically acceptable salt thereof

2. Method according to claim 1 wherein the c-kit inhibitor is selected from Imatinib and Nilotinib or, respectively, a pharmaceutically acceptable salt thereof.

3. (canceled)

4. Method according to claim 2, wherein the Imatinib is applied.

5. Method according to claim 4, wherein the Imatinib is applied in the form of its mono- mesylate salt.

6. Method according to claim 5, wherein the daily dose to be administered is about 300 mg of Imatinib mesylate.

7. Method according to claim 2, wherein the Nilotinib is applied.

8. Method according to claim 7, wherein the Imatinib is applied in the form of its mono- hydrochlonde mono-hydrate salt.

9. Use of a combination comprising a c-kit inhibitor, daunorubicin and cytarabine for the manufacture of a medicament for the treatment of c-kit positive relapsed acute myeloid leukemia AML, wherein the c-kit positive relapsed acute myeloid leukemia AML is observed in patients, which do not show any nuclear P-STAT3 expression.

10. Use according to claim 9, characterized in that the c-kit inhibitor is selected from Imatinib and Nilotinib or a pharmaceutically acceptable salt thereof, respectively.

11. (canceled)

12. (canceled)

13. Package comprising a combination comprising a c-kit inhibitor, daunorubicin and cytarabine together with instructions to use the combination for the treatment of c-kit positive relapsed acute myeloid leukemia AML in patients, which do not show any nuclear P-STAT3 expression.
